(19) United States
(12) Patent Application Publication
(10) Pub. No.: US 2024/0105288 A1
(43) Pub. Date: Mar. 28, 2024

(54) INFERRING DEVICE, TRAINING DEVICE, METHOD, AND NON-TRANSITORY COMPUTER READABLE MEDIUM

(71) Applicant: Preferred Networks, Inc., Chiyoda-ku, Tokyo-to (JP)

(72) Inventors: So TAKAMOTO, Chiyoda-ku, Tokyo-to (JP); Chikashi SHINAGAWA, Chiyoda-ku, Tokyo-to (JP)

(73) Assignee: Preferred Networks, Inc., Chiyoda-ku, Tokyo-to (JP)

(21) Appl. No.: 18/533,481

(22) Filed: Dec. 8, 2023

Related U.S. Application Data
(63) Continuation of application No. PCT/JP22/23521, filed on Jun. 10, 2022.

(30) Foreign Application Priority Data
Jun. 11, 2021 (JP) 2021-098292

(51) Int. Cl. G16C 20/70; G06N 3/045

(52) U.S. Cl. CPC G16C 20/70; G06N 3/045

(57) ABSTRACT

An inferring device includes one or more processors. The one or more processors are configured to acquire an output from a neural network model based on information related to an atomic structure and label information related to an atomic simulation, wherein the neural network model is trained to infer a simulation result with respect to the atomic structure generated by the atomic simulation corresponding to the label information.

### CROSS REFERENCE TO RELATED APPLICATIONS

This application is continuation application of International Application No. JP2022/023521, filed on Jun. 10, 2022, which claims priority to Japanese Patent Application No. 2021-098292, filed on Jun. 11, 2021, the entire contents of which are incorporated herein by reference.

### FIELD

This disclosure relates to an inferring device, a training device, method, and a non-transitory computer readable medium.

### BACKGROUND

Calculation of energy or the like under an environment where a substance is present is widely performed using the first-principles calculation being the atomic simulation. The first-principles calculation calculates the physical property such as energy or the like of an electron system based on the Schroedinger equation, and therefore has high reliability and interpretability. On the other hand, the first-principles calculation takes much calculation time because of successive convergence calculation or the like, and is therefore difficult to apply to exhaustive material search. In contrast to this, a physical property prediction model for a substance using a technique of machine learning such as deep learning is widely developed in recent years. An example of the physical property model is Neural Network Potential (NNP).

In the optimization of this model, supervised learning is often used. It is possible to use, as teacher data, the already acquired result of the first-principles calculation, for example, information acquired from the database or the like published on the web. However, the quantum operation such as the first-principles calculation is realized by approximate calculation based on each technique and parameter, so that the result differs due to a technique to be used, a parameter used in the technique, or the like.

Therefore, for example, even if the NNP is trained using the teacher data acquired by a specific parameter for a specific first-principles calculation technique, the accuracy of deduction is not good due to a change of conditions in some cases. Besides, if the training of the NNP is executed using, as the teacher data, a set of input data and output data acquired by a combination of a plurality of parameters in a plurality of first-principles calculation techniques, there is a problem that the accuracy of the training cannot be improved because the teacher data is not consistent data.

### BRIEF DESCRIPTION OF DRAWINGS

FIG. 1 is a block diagram schematically illustrating an inferring device according to an embodiment.

FIG. 2 is a chart schematically illustrating input/output of a trained model in an inferring device according to an embodiment.

FIG. 3 is a flowchart illustrating processing of an inferring device according to an embodiment.

FIG. 4 is a block diagram schematically illustrating a training device according to an embodiment.

FIG. 5 is a chart schematically illustrating training of a model in a training device according to an embodiment.

FIG. 6 is a flowchart illustrating processing of a training device according to an embodiment.

FIGS. 7 to 9 are charts schematically illustrating a model according to an embodiment.

FIGS. 10 and 11 are flowcharts illustrating processing of a training device according to an embodiment.

FIG. 12 is a chart schematically illustrating a model according to an embodiment.

FIGS. 13 and 14 are flowcharts illustrating processing of a training device according to an embodiment.

FIG. 15 is a diagram illustrating one example of an implementation of an information processing device according to an embodiment.

### DETAILED DESCRIPTION

According to one embodiment, an inferring device includes one or more processors. The one or more processors are configured to acquire an output from a neural network model based on information related to an atomic structure and label information in an atomic simulation, wherein the neural network model is trained to infer a simulation result with respect to the atomic structure generated by the atomic simulation corresponding to the label information.

Hereinafter, embodiments of the present invention will be explained with reference to the drawings. The drawings and the explanation of the embodiments are indicated as examples and are not intended to limit the present invention.

### First Embodiment

First of all, some terms in this disclosure will be explained.

Interatomic potential (interaction potential energy between atoms) is a function for finding energy from the arrangement of atoms. This function is generally an artificial function. This is a function corresponding to a governing equation for performing Molecular Dynamics (MD) simulation. A non-limiting example of the interatomic potential is Lennard Jones potential.

Neural Network Potential (NNP) expresses the interatomic potential by a neural network.

A 2-body potential curve shows the relation between a distance between two atoms and energy in the case where only the two atoms are present in a system.

Density Functional Theory (DFT) is a technique of calculating a physical state with respect to the structure of an atom according to the Schroedinger equation. The DFT is extremely high in calculation load but can acquire a highly accurate result. In training the NNP, for example, training data is generated by an arithmetic operation based on the DFT.

The Schroedinger equation has difficulty in finding an exact solution except in special cases. Therefore, the DFT numerically analyzes the Schroedinger equation and acquires a solution by approximate calculation. There are a plurality of techniques of the approximate calculation in the DFT and there are suitable situations for them respectively, so that various approximate techniques are practically used. Depending on the approximate techniques, different calculation results are highly likely to be acquired. This approximate calculation algorithm is selected depending on how strict the accuracy should be taken, whether a specific phenomenon should be taken into consideration, what should be used for a functional (empirical function), or the like.

Examples of software for performing the arithmetic operation of the DFT include VASP (registered trademark), Gaussian (registered trademark), and so on. These use different approximation algorithms. For example, VASP is considered to be high in accuracy with respect to a periodic boundary condition, and Gaussian is considered to be high in accuracy with respect to a free boundary condition. The periodic boundary condition is a structure which infinitely (in a sufficiently large range) continues such as a crystal, and the free boundary condition is a structure in which a molecule is isolated in vacuum. In the above example, it is desirable to use VASP when the arithmetic operation is desired to be executed for a crystal or the like, and to use Gaussian when the arithmetic operation is desired to be executed for the structure in which a molecule or the like is isolated.

Though examples where the DFT is used in the first-principles calculation and VASP and Gaussian are used as the DFT will be explained in some embodiments, the content of this disclosure is not limited to them but can be applied to various techniques. Besides, the simulation result to be acquired will be explained using potential information (information related to energy, force, and the like), but can be similarly realized even using other information according to other algorithms.

(Inferring Device)

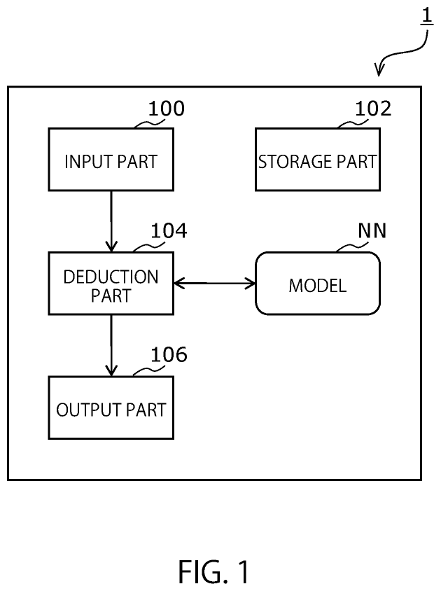

FIG. 1 is a block diagram schematically illustrating an inferring device according to an embodiment. An inferring device 1 includes an input part 100, a storage part 102, a deduction part 104, and an output part 106. The inferring device 1 is a device which executes deduction based on the NNP which outputs information related to a potential when an atomic structure on a compound, an environment, and the like is input.

The input part 100 is an interface which accepts input of data in the inferring device 1. The inferring device 1 acquires information or the like (hereinafter, described as an atomic structure) on a compound whose potential information is desired to be acquired via the input part 100. The atomic structure may include, as an example, information related to the type and position of an atom. Examples of the information related to the position of an atom include information directly representing the position of an atom by coordinates, information directly or indirectly representing the relative positions between atoms, and so on. Further, the information related to the position of an atom may be information expressing the positional relation between atoms by a distance, an angle, a dihedral angle, or the like between atoms. The atomic structure may further include information related to a boundary condition. Further, the inferring device 1 can receive input of software which uses an algorithm for acquiring the potential information via the input part 100 and information (hereinafter, described as label information) related to a value of a parameter when using the software.

The storage part 102 stores various types of data required for processing of the inferring device 1. For example, the storage part 102 may temporarily store the information related to a compound input from the input part 100, and store a hyperparameter, parameter, and the like for implementing a trained model.

The deduction part 104 inputs the atomic structure and the label information which are input via the input part 100 into the model NN and thereby acquires the potential information related to the atomic structure calculated based on the label information. The deduction part 104 may convert the data format input from the input part 100 into a data format to be input into an input layer of the model NN as necessary.

The model NN is a trained neural network model and is, for example, a model to be used for acquiring the potential in the NNP. The information for forming the model NN may be stored in the storage part 102 and the model NN may be formed in executing the deduction. The model NN may be an arbitrary neural network model which can appropriately perform input/output in this embodiment and may be, for example, a neural network model including a convolution layer and a fully connected layer, a neural network model including Multi-Layer Perceptron (MLP), or a neural network model capable of handling a graph.

The output part 106 outputs a result deducted by the deduction part 104 using the model NN to an external part or the storage part 102.

An example of the data input/output to/from the inferring device 1 will be explained.

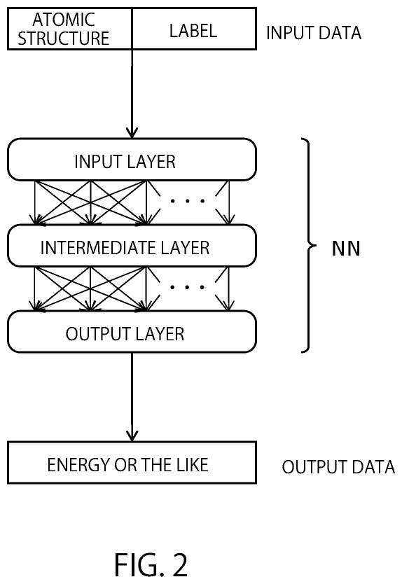

FIG. 2 is a chart schematically illustrating input/output data in the model NN of the inferring device 1 according to an embodiment. For example, the model NN receives the atomic structure and the label information input into the input layer, forward propagates the input data, and outputs energy from an output layer. The model NN includes one intermediate layer in this chart, but may include many intermediate layers. For example, the model NN may be configured to include many layers trained by deep learning.

The deduction part 104 of the inferring device 1 may acquire information related to force by performing position differentiation (finding a gradient with respect to a position) using the positional information input as the atomic structure, on the energy output from the model NN. It becomes possible to acquire differential information, for example, by acquiring the output from the model NN while slightly shifting the positional information in the atomic structure to be input. Besides, the information related to the force may be acquired by backward propagating the energy to the model NN.

This model NN is trained by a later-explained training device, and therefore outputs the energy or the like based on the label information by receiving input of input data including the atomic structure and the label information. In other words, by designating what parameter is to be used for which algorithm (software) for calculating energy with respect to a certain atomic structure, it is possible to output an inferred value in the algorithm and parameter desired by the user from the output layer.

Note that it is also adaptable that the deduction part 104 inputs appropriate algorithm, parameter, and the like as the label information into the model NN based on the condition of the atomic structure or the like without designation by the user, and outputs a desired, for example, highly accurate result. Further, also in the case where the user designates the label information, it is adaptable to select the label information determined by the deduction part 104 to provide higher accuracy, and output the result designated by the user and the result selected by the deduction part 104 together. Examples of the highly accurate result include the one to which a label related to VASP is attached under the periodic boundary condition and the one to which a label related to Gaussian is attached under the free boundary condition, but not limited to these examples.

Besides, in training, a neural network model which connects the atomic structure and the label information may be trained separately from the model NN. This neural network model is, for example, a model which outputs the label information when the atomic structure is input. This neural network model can output, for example, the label information often added to similar atomic structures in a training data set. The deduction part 104 may input the atomic structure into this neural network model, acquire the label information, and input the atomic structure and the output label information into the model NN.

Besides, as is known from the description in the previous paragraph, it is also adaptable not to form the neural network model, but to acquire some statistical information with respect to the atomic structure and add the label information based on the statistical information on a rule basis.

In any of the above cases, the inferring device 1 may output the selected label information together with the potential information when the deduction part 104 decides the label information.

The configuration of the input data will be explained later in detail together with the configuration of the model NN.

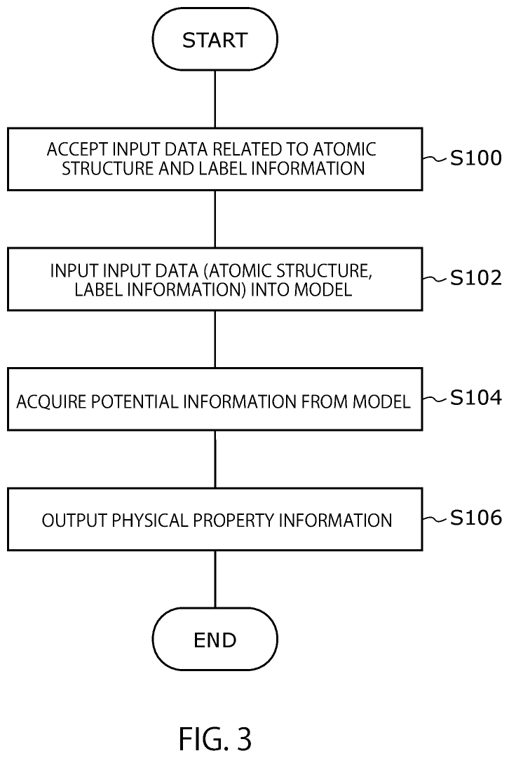

FIG. 3 is a flowchart illustrating processing of the inferring device 1 according to an embodiment.

The inferring device 1 accepts data on a label structure including an atomic structure and information on an algorithm to be applied to the atomic structure via the input part 100 (S100). If necessary, the inferring device 1 stores the input data in the storage part 102.

The deduction part 104 inputs the above input data including the atomic structure and the label information into the model NN and forward propagates the input data (S102). In the case where the input data is not in a format suitable for input into the model NN, the deduction part 104 converts the input data into a format suitable for input into the model NN, and inputs the converted input data into the model NN.

The deduction part 104 acquires the result obtained by the forward propagation from the model NN (S104). The result obtained by the forward propagation is data including the acquired potential information.

The inferring device 1 outputs, via the output part 106, the potential information acquired by the deduction part 104 (S106).

The use of the inferring device according to this embodiment as explained above makes it possible to acquire the potential information in the first-principles calculation with designated software. As a result of this, it becomes possible to infer the result using various algorithms for various structures. Further, it also becomes possible to perform inference with different parameters in the software. For example, even in the case where an approximate solution cannot be appropriately acquired by the DFT, the inferring device according to this embodiment can appropriately acquire the approximate solution and can acquire the potential information with high generalization performance or robustness.

(Training Device)

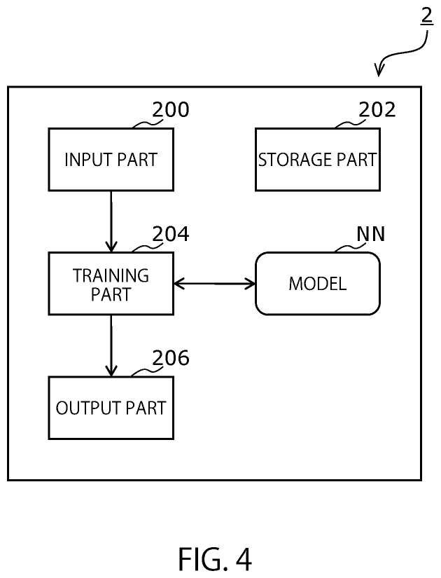

FIG. 4 is a block diagram schematically illustrating a training device according to an embodiment. A training device 2 includes an input part 200, a storage part 202, a training part 204, and an output part 206. This training device 2 is a device which executes deduction based on the NNP which outputs the potential information when an atomic structure on a compound, an environment, and the like is input. Further, the training device 2 executes training so as to be able to receive input of the information related to software in the deduction of the NNP similarly to the input data into the model NN in the above inferring device 1.

The input part 200 is an interface which accepts input of data in the training device 2. The training device 2 accepts training data (teacher data) including information on an atomic structure, label information and the atomic structure, and potential information calculated based on a label structure, as the input data via the input part 200.

The storage part 202 stores various types of data required for processing of the training device 2. For example, the storage part 202 may store a combination of the potential information and the atomic structure and label information input from the input part 200, and use it in training. Further, the storage part 202 may store the parameter and the like in training. In the training device 2, the data to be used for training is generally large in amount, and therefore the storage part 202 does not need to be provided in the same housing as that in which other components of the training device 2 are provided. For example, at least a part of the storage part 202 may be provided in a file server via a communication path. In this case, the acquisition of the data from the file server or the like may be executed via the input part 200.

The training part 204 inputs the atomic structure and the label information which are the training data into the NN being the neural network model to acquire output data. The training part 204 compares the potential information connected with the atomic structure and the label information and the output data from the model NN to calculate an error, and updates the parameter based on the error. This training is not particularly limited, but may be executed using a general machine learning technique or a deep learning technique. For example, the training part 204 may backward propagate the output error, calculate a gradient of a weighting matrix or the like between layers constituting the model NN based on the backward propagated error, and update the parameter using this gradient.

The output part 206 outputs the parameter or the like related to the model NN optimized by training by the training part 204 to the external part or the storage part 202.

In the above inferring device 1, the model NN needs to output the potential information based on the atomic structure and the label information. Therefore, the training device 2 trains the model NN so as to output the potential information calculated from the atomic structure based on the algorithm (software) included in the label information and information on an arithmetic parameter.

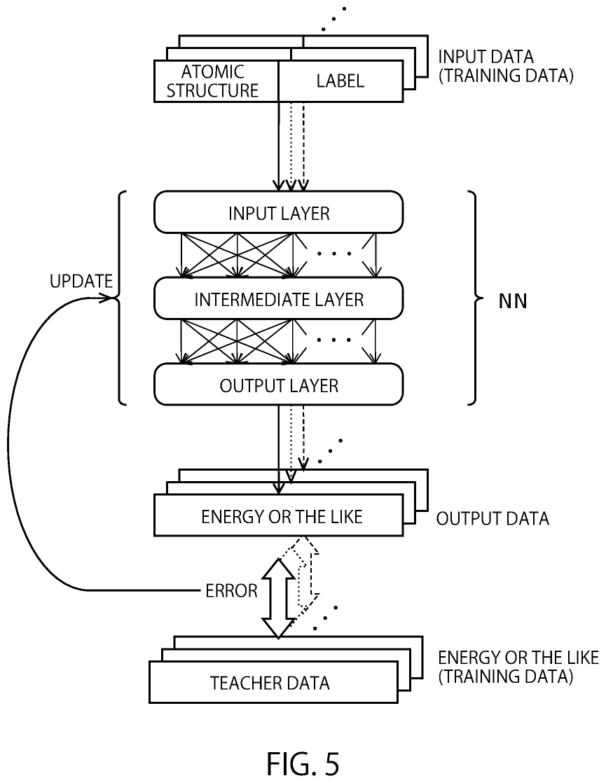

FIG. 5 is a chart schematically illustrating an example of training of the model NN in the training device 2. The training part 204 inputs the data related to the atomic structure and the label information of the input data set into the model NN. The training part 204 calculates an error between the potential information on the energy or the like output from the model NN and the potential information on the energy or the like acquired by a predetermined arithmetic technique based on the input data corresponding to each output. The training part 204 then updates the parameter of the like of the model NN using the error to thereby execute training.

The label information includes at least the software used for finding the potential of the energy or the like from the atomic structure and the information on the arithmetic parameter or the like used for finding the potential information in the software as explained above. The training data is data including the atomic structure and the label information, and an appropriately large mount of the data is required as in the general machine learning.

In the training device 2 according to this embodiment, it is desirable that pieces of data belonging to different domains, namely, a plurality of pieces of data having different pieces of label information are prepared as the training data. Further, it is more desirable that pieces of data related to various atomic structures exist in the same label information.

The training device 2 does not train the model NN while separating the pieces of training data for each piece of label information, but executes training in a state of mixing the pieces of training data irrespective of the label information. For example, in the case of executing training by mini batch processing as the technique of machine learning, the training device 2 preferably performs training using data having different pieces of label information in a batch.

However, all the execution of training using the training data composed of the same label information is not excluded. For example, if the parameters using finally different pieces of label information are updated in the training in the training device 2, a mini batch having only the same label information may exist.

Besides, it is desirable that in the different pieces of label information, a common atomic structure and values of energy or the like in the pieces of label information with respect to the common atomic structure are provided as data. For example, even if the approximate technique is different, it is presumed that there is linear or non-linear relevance in output data between the different pieces of label information.

If the data related to the common atomic structure is not present in the different pieces of label information, the neural network model is trained so as to match the training data acquired in each piece of label information, so that it may fail to perform appropriate training and deduction on intermediate information of the pieces of label information with respect to the common atomic structure.

To cope with the above, it is desirable to use the training data including data on the energy or the like about the same atomic structure or atomic structures belonging to the same environment with respect to the different pieces of label information. The use of the training data having the same atomic structure or the like reflects the above linear or non-linear relevance in the training. As a result of this, it becomes possible to execute appropriate deducing processing even if the atomic structure in a similar environment with respect to the label information does not exist but if data on the similar atomic structure exists as the training data in other piece of label information.

As explained above, in this embodiment, it is possible to improve the accuracy in deduction using the model NN by executing training using the training data including the same atomic structure or an atomic structure (almost the same atomic structure) which can be regarded as the same.

The same atomic structure is, as a non-limiting example, an atomic structure of the same substance, and almost the same atomic structure is, as a non-limiting example, an atomic arrangement of substances in which the substances are different but similar points are recognized in configuration such as the same atomic arrangement with the same molecular weight, atomicity, and different atoms. Besides, when there are molecules at positions separated from a crystal, the case where the distance between the molecule and the molecule or between the crystal and the molecule and their postures are different may also be regarded as almost the same atomic structure.

A concrete example when finding energy from the atomic structure will be explained. It is assumed that first software is VASP and second software is Gaussian. It is assumed that a first condition is a condition of applying an appropriate parameter for VASP and a second condition is a condition of applying an appropriate parameter for Gaussian. First label information is information including the first condition and second label information is information including the second condition.

VASP is software using the DFT being the first-principles calculation and is high in accuracy when setting the periodic boundary condition suitable for expressing the structure of a crystal as the boundary condition of the atomic structure. Therefore, VASP can calculate appropriate energy for a substance such as a crystal.

On the other hand, Gaussian is software using the DFT being the first-principles calculation, and is high in accuracy in the case of setting the free boundary condition which is suitable for expressing a structure in which a molecule or the like is isolated in vacuum, as the boundary condition of the atomic structure. Therefore, Gaussian can calculate appropriate energy for a substance such as a molecule.

Therefore, it is possible to collect the information on energy related to various crystal structures acquired under the first condition and the data on energy related to various molecular structures acquired under the second condition as highly accurate data, as the training data.

On the other hand, in this embodiment, the structure being an intermediate region between these atomic structures is also acquired by parameter setting based on the label information both in VASP and Gaussian. As this intermediate data, for example, data on a region where a result at a certain level of accuracy in the approximate calculation is acquired in any of VASP and Gaussian for an atomic structure indicating a molecule with a unit size of a space set up to about 10 Å, an atomic structure indicating the crystal structure and a molecule existing at a position sufficiently distant from a surface of a crystal with a sufficiently large unit size of a space, an atomic structure having a free boundary condition in which the number of atoms reaches several of hundreds, or the like in the atomic structure of the molecule. As explained above, it becomes possible to acquire pieces of training data different in label information in the same (or almost the same) atomic structure.

As explained above, through use of the common atomic structure under the first condition and the second condition, the training device 2 trains the model NN about the relevance between the first condition and the second condition. As this training result, the relevance between the first condition and the second condition is incorporated into the model NN, thereby making it possible to train the model NN which can infer the amount of energy or the like about “the atomic structure suitable for calculation under the second condition” under the first condition for instance.

Note that though VASP and Gaussian are exemplified for acquiring the potential information in the above, the software to be used is not limited to them. The software only needs to be the one which performs approximate calculation using different algorithms and, for example, other software such as GAMESS, WIEN2k, PHASE, CASTEP, or Quantum Espresso may be used. Further, not the software using the DFT but software which can realize the first-principles calculation using another technique may be used. For example, software which executes an arithmetic operation based on the Hartree-Fock method, the MP2 method, or the like may be used. Furthermore, software which executes not the first-principles calculation but another atomic simulation for acquiring a simulation result may be used.

Also in these cases, it is desirable to acquire the training data on the same (or almost the same) atomic structure in a combination of the software to be used and the parameter.

In summary, the training device 2 calculates a first error between a first result output by inputting data related to a first atomic structure and the first label information including the first condition into the model NN and a first simulation result obtained by approximate calculation under the first condition (a certain parameter in first software (first algorithm may be used)) for the first atomic structure, and uses the first error for training of the model NN.

Similarly, the training device 2 calculates a second error between a second result output by inputting data related to a second atomic structure and the second label information including the second condition into the model NN and a second simulation result obtained by approximate calculation under the second condition (a certain parameter in second software (second algorithm may be used)) for the second atomic structure, and uses the second error for training of the model NN.

Note that the use of the first algorithm in the first software and the use of the second algorithm in the first software can also be regarded as separate conditions.

The first software included in the first condition and the second software included in the second condition are pieces of software which can acquire the same type of potential information. For example, these pieces of software are each software which calculates potential (energy) by the first-principles calculation. Further, the DFT may be used for the first-principles calculation. Further, these pieces of software may acquire information on force related to a substance. The training part 204 may perform position differentiation on the value of energy output from the model NN to further acquire the information on force, and may execute update of the parameter using this information.

For example, the first condition may be a condition under which an arithmetic operation higher in accuracy than under the second condition can be executed when using the periodic boundary condition. Further, the second condition may be a condition under which an arithmetic operation higher in accuracy than under the first condition can be executed when using the free boundary condition. As a non-limiting example which satisfies them, the first software used under the first condition may be VASP and the second software used under the second condition may be Gaussian.

The training data desirably includes a data set of a plurality of first atomic structures with respect to the first label information and the first simulation results corresponding to the first atomic structures and a data set of a plurality of second atomic structures with respect to the second label information and the second simulation results corresponding to the second atomic structures. Further, the data set of the first atomic structures and the data set of the second atomic structures desirably include the same or almost the same (data belonging to the same domain related to the atomic structure) atomic structures. As a matter of course, the first simulation result and the second simulation result with respect to the same or almost the same atomic structure are results obtained by arithmetic operations with different algorithms and parameters, and therefore may indicate different energy values.

As another example, both of the first software and the second software are VASP, and separate calculation techniques or parameters may be used as the first condition and the second condition. Further, the first condition and the second condition may be the same in software and different both in calculation technique and parameter.

In other words, the label information can include various types of information on the calculation technique, the function used for the calculation technique, the parameter in the calculation technique, and so on. A simulation may be executed based on the above information to generate the data set to be used for training. As a non-limiting example, different calculation conditions or the same calculation condition by different pieces of software, different calculation conditions or the same calculation condition by the same software, or the like may be executed in an arbitrary combination in a range in which a simulation can be executed to generate the data set. The use of the above data set makes it possible to realize the training of a model higher in accuracy with respect to an input with the label information added thereto.

The training device 2 executes the training of the model NN with the training data including the above information and thereby can realize optimization of the model NN improved in generalization performance.

Note that though a first . . . and a second . . . are exemplified in the above, there may be a third . . . , a fourth . . . , and so on as a matter of course. The number of them is not limited. Further, it is desirable that the same relevance as in the above is secured also for the third . . . , the fourth . . . , and so on. For example, the condition is not limited to the two conditions such as the first condition and the second condition but there may be three or more conditions. The label information is not limited to two pieces of label information such as the first label information and the second label information, but there may be three or more pieces of label information. The atomic structure is not limited to the two atomic structures such as the first atomic structure and the second atomic structure, but there may be three or more atomic structures. The simulation result is not limited to the two simulation results such as the first simulation result and the second simulation result, but there may be three or more simulation results. The neural network model may be trained by the same method as above based on these pieces of information.

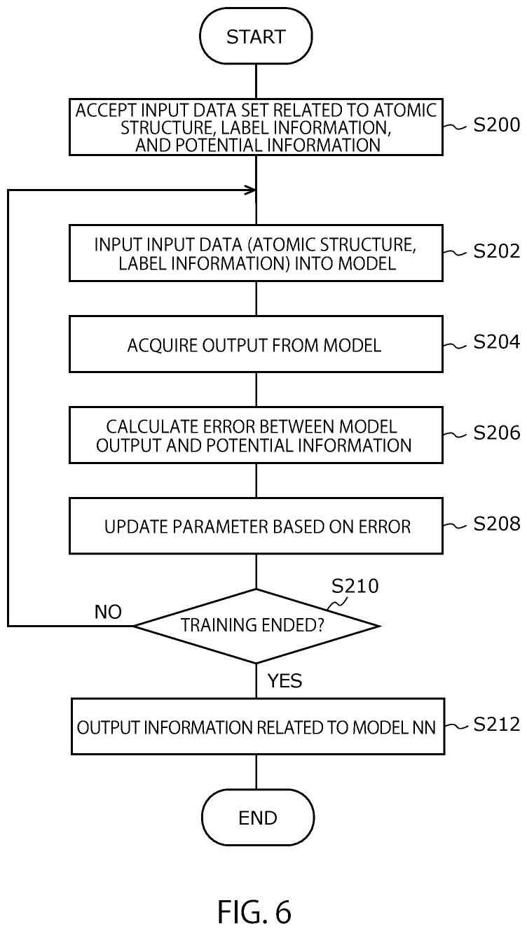

FIG. 6 is a flowchart illustrating processing of a training device according to an embodiment.

The training device 2 accepts the training data via the input part 200 (S200).

The training part 204 inputs the data related to the atomic structure and the data related to the label information of the input training data into the model NN, and forward propagates them (S202). If the input data is not in a format suitable for input into the model NN, the training part 204 converts the input data into a format suitable for input into the model NN and inputs the converted input data into the model NN.

The training part 204 acquires a result of the forward propagation from the model NN (S204). This result of the forward propagation is data including information desired to be acquired as the potential information.

The training part 204 compares the information acquired from the model NN and the potential information corresponding to the data input into the model NN to calculate an error (S206).

The training part 204 updates the parameter of the model NN based on the error (S208). The training part 204 updates the parameter of the model NN, for example, based on the gradient by the error backpropagation method.

The training part 204 determines whether the training has been ended based on an end condition set in advance (S210). The end condition may be equal to an end condition of the general machine learning technique.

If the end condition of the training is not satisfied (S210: NO), the processing from S202 is repeated. If necessary, the training data to be input into the model NN is changed and the processing from S202 is repeated.

If the end condition of the training is satisfied (S210: YES), the trained data required for construction of the model NN such as the parameter of the model NN is appropriately output and the processing is ended (S212).

As explained above, this model NN is trained as the neural network model which acquires a first output (for example, a result by the first-principles calculation) obtained by inputting the information related to the first atomic structure and the first label information related to the first condition into the neural network model when the information related to the first atomic structure and the first label information are input and acquires a second output (for example, a result by the first-principles calculation) when the information related to the second atomic structure and the second label information are input, and is used in the inferring device 1.

As explained above, the use of the training device according to this embodiment makes it possible to train the neural network model which can realize deduction in consideration of the software, the arithmetic parameter, and so on. The trained model trained by the training device can perform deduction improved in generalization performance with respect to the software and the arithmetic parameter.

For example, a domain where an arithmetic operation is performed by VASP and a domain where an arithmetic operation is performed by Gaussian are generally different, but the use of the model trained as above makes it possible to acquire a result obtained by an arithmetic operation by Gaussian in the domain where it is better to perform the arithmetic operation by VASP. For example, it is possible to generate a model which infers energy of a crystal using Gaussian suitable for energy acquisition of a molecule.

The use of this model as the above model NN in the inferring device 1 allows the user to acquire the potential information on energy or the like with the designated software and arithmetic parameter. For example, in the case where the user desires to compare the energy value between a molecular domain and a crystal domain, it becomes possible to compare not results using different approximate calculation techniques but results using the pseudo-same approximate calculation technique.

Next, the input data into the neural network model in this embodiment will be explained using some non-limiting examples. In the inferring device 1 and the training device 2 according to this embodiment, the data input into the model NN includes the atomic structure and the label information.

The atomic structure includes, as an example, information related to the boundary condition and information related to a constituting atom. A vector related to the boundary condition is assumed to be B, and a vector related to the constituting atom is assumed to be A. In this case, a vector C indicating the atomic structure can be expressed as follows by concatenating B and A.

C=[B,A]

The information related to the boundary condition is information indicating the free boundary condition and the periodic boundary condition. Further, the case of the periodic boundary condition includes information indicating the size of a unit indicating the atomic structure. For example, the information related to the boundary condition can be expressed as follows.

B=[Btype,Bx,By,Bz]

Btype is a binary value indicating the free boundary condition or the periodic boundary condition. Bx, By, Bz express the unit size in the case of the periodic boundary condition, using three axes. For example, it is assumed that the case of the free boundary condition is Btype=0 and the case of the periodic boundary condition is Btype=1. Further, in the case of the periodic boundary condition, the unit size is designated to Bx, By, Bz. In order to avoid noise in the training, in the case of the free boundary condition, all of Bx, By, Bz may be set to 0. In addition, in the deduction part 104 and the training part 204, a product of Btype (0 in the case where the free boundary condition is designated) and each of Bx, By, Bz may be input into the model NN.

Besides, the following is adaptable without using Btype.

B=[Bx,By,Bz]

It is adaptable that Bx=By=Bz=0 in the case of designating the free boundary condition, and the unit size is Bx, By, Bz in the case of designating the periodic boundary condition. The unit of Bx, By, Bz may be Å. For example, an origin is set, and the lengths of Bx in an X-axis direction, By in a y-axis direction, and Bz in a z-axis direction from the origin are designated as the unit size. The positional information on an atom can be designated as the positional information (coordinate information) with respect to the origin.

Besides, the vector B may include a parameter indicating the shape of the unit. The vector B may further include three elements indicating angles of the three axes, and may further include an element related to the other shape.

The information related to the constituting atom is set for each of atoms constituting a substance with the type of the constituting atom and the positional information on the atom as a set. For example, there are atoms Atom1, Atom2, . . . , AtomN, the information can be expressed as follows.

A=[Atom1t,Atom1x,Atom1y,Atom1z,Atom1t,Atom2x,Atom2y,Atom2z, . . . ,AtomNt,AtomNx,AtomNy,AtomNz]

AtomXt indicates the type of an atom of AtomX. The type of the atom may be indicated, for example, by an atomic number such as 1 for a hydrogen atom and 6 for a carbon atom.

AtomXx, AtomXy, AtomXz each indicate the position where AtomX is present. As explained above, this position may be indicated by the coordinates from the origin using A as a unit and may be indicated by coordinates using another base unit, and is not limited to these descriptions.

It is assumed that in the case where there are N atoms, a vector obtained by concatenating N pieces of information on the above AtomXt, AtomXx, AtomXy, AtomXz is A.

In other words, the vector C indicating the atomic structure is expressed as follows.

C=[Btype,Bx,By,Bz,Atom1t,Atom1x,Atom1y,Atom1z,Atom2t,Atom2x,Atom2y,Atom2z, . . . ,AtomNt,AtomNx,AtomNy,AtomNz]

In addition to the above, a variable designating the number of atoms may be included.

Next, a vector L indicating the label information will be explained. The label information includes software used for inference in the inferring device 1 or for acquiring the training data in the training device 2, and a parameter used in the software. The software is described here but may be read as algorithm. It is assumed that a vector (or scalar) indicating the software is S and a vector indicating the parameter is P. The label information L may be defined as follows by concatenating S and P.

L=[S,P]

S may be a scalar expressed as 1 when using VASP and 2 when using Gaussian. In this case, in the deduction, a virtual approximation arithmetic unit 1.5 between VASP and Gaussian or the like can also be designated. As another example, when using three or more pieces of software, S can be designated as 3, 4, . . . or the like.

Besides, as another example of a vector expression, the following equation or the like may be used.

S=[V,G]

A one-hot vector format such as S=[1, 0] when the software to be used is VASP and S=[0, 1] when the software to be used is Gaussian can be used. The case of using furthermore pieces of software in training/deduction can be handled by lengthening the one-hot vector.

P is expressed by a vector designating a parameter to be used in each piece of software. For example, P can be expressed as follows in the case of using M pieces of parameter information.

P=[Param1,Param2, . . . ,ParamM]

Each element of the vector may be in any of expressions of a discrete value (including an integer value), a toggle value, and a continuous value.

In the case where each parameter is expressed by a discrete value, P can be expressed by the following one-hot vector.

P=[Param1_1,Param1_2, . . . ,Param1_1,Parm2_1, . . . ,Param2_j, . . . ,ParamM_1, . . . ,ParamM_k]

Besides, P may be expressed as follows with a part thereof expressed by one-hot vector.

P=[Param1,Param2_1, . . . ,Param2_j, . . . ,ParamM]

As a concrete example of the label information, the following arithmetic mode is considered. It is assumed that the mode can be expressed as {software, exchange-correlation functional, basis function, with/without using DFT+U} as a simple example.
Mode 1: {Gaussian, ωB97XD, 6-31G(d), without}
Mode 2: {VASP, PBE, plane wave, with}
Mode 3: {VASP, PBE, plane wave, without}
Mode 4: {VASP, rPBE, plane wave, without}

In the case of the above mode setting (label information), L can be expressed by a vector (a scalar indicating software and a three-dimensional vector indicating parameters) having four elements. As a matter of course, an arbitrary element may be expressed by the one-hot vector as explained above.

For example, it is assumed that as the software information, VASP is 1 and Gaussian is 2. In the parameter information, ωB97XD is 1, PBE is 2, and rPBE is 3 as the exchange-correlation functional, and 6-31G(d) is 1 and the plane wave is 2 as the basis function, and the case of using DFT+U is 1 and the case of not using DFT+U is 0 as DFT+U. With the above definition, each mode can be re-written as follows.
Mode 1: L=[2, 1, 1, 0]
Mode 2: L=[1, 2, 2, 1]
Mode 3: L=[1, 2, 2, 0]
Mode 4: L=[1, 3, 2, 0]

Note that DFT+U can be designated as a continuous value. In this case, it is adaptable that DFT+U is not used for 0 and a continuous value indicating a parameter related to DFT+U is used for other than 0.

The above mode can be rewritten as follows when describing the software by a one-hot vector.
Mode 1: L=[0, 1, 1, 1, 0]
Mode 2: L=[1, 0, 2, 2, 1]
Mode 3: L=[1, 0, 2, 2, 0]
Mode 4: L=[1, 0, 3, 2, 0]

The examples of the above-explained parameters and various expression methods are merely examples, and do not limit the technical scope of this disclosure. Expression methods of vector, matrix, or tensor in various evolutionary arbitrary dimensions can be used.

The training device 2 acquires the output by inputting the atomic structure and the label information defined as above into the model NN, compares the acquired output and the potential information in the training data, and updates the parameter of the model NN.

Then, the inferring device 1 can acquire the potential information subjected to an arithmetic operation based on the label information by inputting the label information (for example, in the above mode) and the atomic structure using the model NN trained as above.

Note that as the interface for input/output, the inferring device 1 may have a form which causes the user to select the information related to the aforementioned mode. In this case, the user inputs the atomic structure for which the user desires to acquire the potential information and selects the mode, and thereby can acquire the potential information corresponding to the atomic structure which has been subjected to the arithmetic operation in the selected mode.

Note that the label information in this embodiment only needs to include information related to at least one of various calculation conditions in the atomic simulation, calculation technique (calculation algorithm algorithm), software to be used for calculation, and various parameter in the software. Besides, the first condition and the second condition in the atomic simulation may be a condition in which at least one of the above pieces of label information is different. Besides, though the first-principles calculation is indicated as one example of the atomic simulation in this embodiment, the simulation result may be acquired using other techniques. The atomic simulation may be executed using a semi-empirical molecular orbital method, a fragment molecular orbital method, or the like to acquire the simulation result.

According to this embodiment, it is possible to generate a model which can appropriately acquire the potential information on the atomic structure based on the label information and realize the deduction based on this model, for the atomic structure with the label information added thereto. In the DFT calculation, the accuracy may differ even for the same atomic structure, depending on the calculation condition. According to the training and deduction in this embodiment, it is possible to perform training and deduction while designating the calculation technique irrespective of a domain. Therefore, the NNP using the model according to this embodiment can acquire the result under the appropriate calculation condition in an appropriate domain. Further, even in the case of not an appropriate (high in accuracy) domain with respect to the calculation condition, it is possible to perform such training that corrects the difference between the calculation condition and the other calculation condition. Therefore, applying the training and deduction according to this embodiment to the model used for the NNP makes it possible to appropriately infer pieces of potential information on the atomic structures belonging to various domains under various calculation conditions.

More specifically, the result of the DFT calculation tends to have a deviation in output due to the software, parameter, or the like with respect to the same input. On the other hand, the result itself of the DFT calculation is generally uniquely decided, and therefore the deviation affects the training in the training of the NNP model. For example, in the atomic structures belonging to the same domain, the calculation results are different due to software and the results themselves have no noise, so that the training is performed using the teacher data having a plurality of solutions with respect to the same atomic structure. Therefore, the training of the model is unstable in the state without a label.

As compared with the above, the training is performed while adding the label information as in this embodiment, whereby the model can perform learning while clearly distinguishing the deviation in the result between a plurality of pieces of software. Therefore, as explained above, the training and deduction according to this embodiment have great effects in the NNP. Further, it is possible to improve the generalization performance by adding variations of the data set about the calculation technique and the atomic structure.

### Second Embodiment

The atomic structure and the label information are configured to be input into the input layer of the model NN in the first embodiment, but are not limited to this configuration.

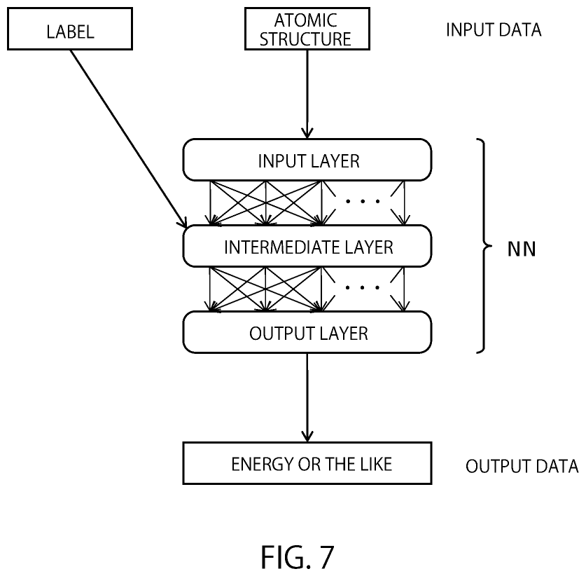

FIG. 7 is a chart illustrating an example of a model NN according to this embodiment. As illustrated in FIG. 7, the model NN may be configured to receive input of the atomic structure at the input layer while receiving input of the label information at an arbitrary intermediate layer. An arbitrary bias may be applied to the label information, and the bias may also be trained by the training device 2 as with the weight between layers.

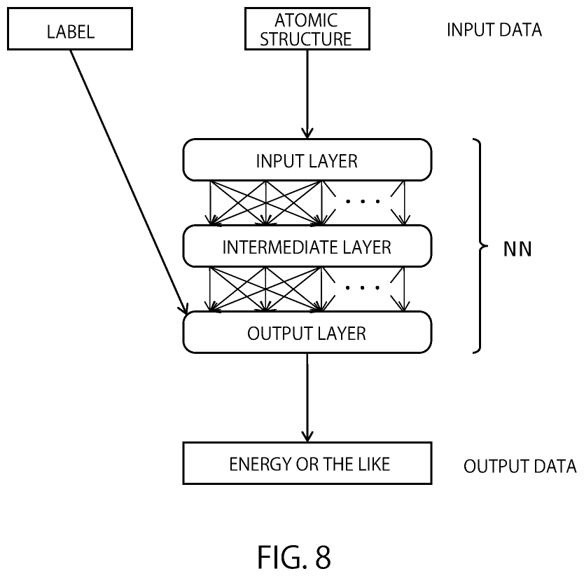

FIG. 8 is a chart illustrating another example of the model NN according to this embodiment. As illustrated in FIG. 8, the model NN may be configured to receive input of the atomic structure at the input layer while receiving input of the label information at the output layer. Also in this case, an arbitrary bias may be applied to the label information.

In the case where the model NN has the configurations in FIG. 7 and FIG. 8, the training can be executed by forward propagating the atomic structure at S202 in FIG. 6 and inputting the label information at an appropriate intermediate layer or the output layer.

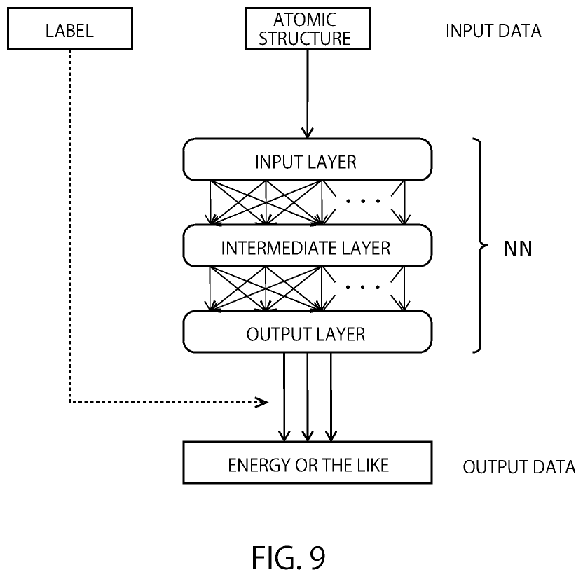

FIG. 9 is a chart illustrating another example of the model NN according to this embodiment. The model NN illustrated in FIG. 9 has a configuration which outputs an output corresponding to a plurality of pieces of label information from the output layer when the atomic structure is input.

The training device 2 performs training, for example, so that the potential information is output from a node corresponding to the label information in the output layer when the atomic structure is input. The outputs from the other nodes are ignored, for example, in the training.

In the case where pieces of potential information collected in different pieces of label information exist as the training information with respect to the same atomic structure, the output from the model NN and the potential information (teacher information) corresponding to the label information are compared for each node corresponding to the label information, and the parameter of the model NN is updated based on the comparison result.

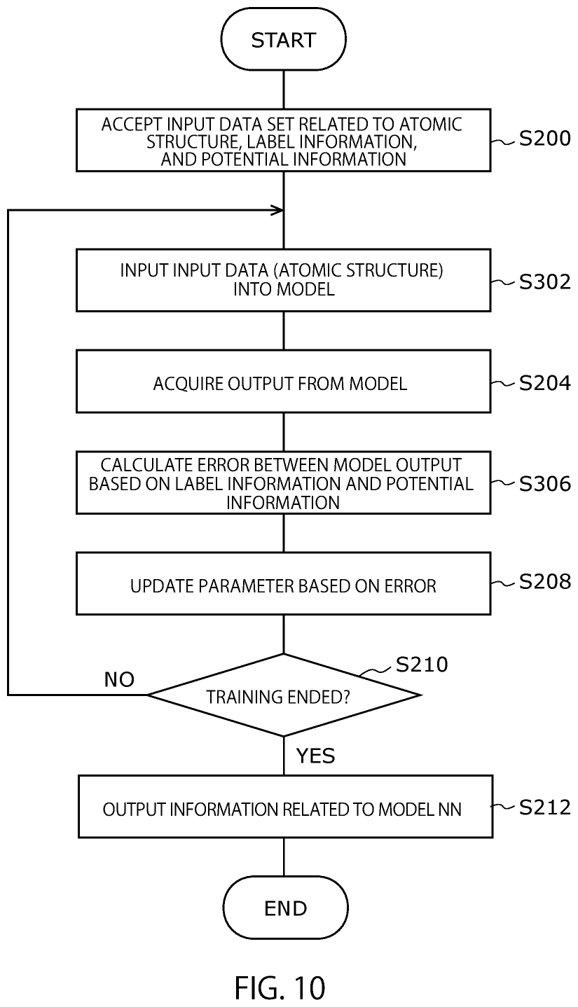

FIG. 10 is a flowchart illustrating processing of training by the training device 2 in the configuration of FIG. 9. Processing with the same code as that in FIG. 6 represents the same processing.

When a data set is input, the training part 204 inputs the information related to the atomic structure into the input layer of the model NN (S302). The training part 204 executes forward propagation in the model NN to acquire results of the forward propagation corresponding to the plurality of pieces of label information from the output layer (S204).

The training part 204 acquires an output value corresponding to the label information in the data set used for the training of the output result, and calculates an error between the output value corresponding to the label information and the potential information (S306).

The training part 204 then updates the parameter of a model NN2 based on the error (S208). In this case, pieces of potential information corresponding to a plurality of pieces of label information are output from the output layer, but if the label information related to the input atomic structure does not exist, the backward propagation processing does not need to be executed from the corresponding node of the output layer. Besides, if a plurality of pieces of label information related to the input atomic structure exist, the backward propagation may be executed from the node of the output layer corresponding to each of the pieces of label information.

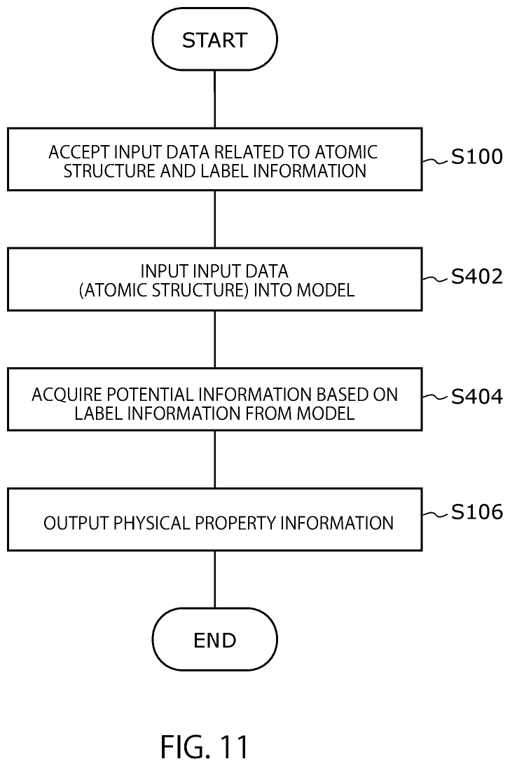

FIG. 11 is a flowchart illustrating inferring processing of the inferring device 1 in the configuration in FIG. 9. Processing with the same code as that in FIG. 3 represents the same processing.

The deduction part 104 inputs the atomic structure into the input layer of the model NN (S402).

The deduction part 104 forward propagates it through the model NN to acquire pieces of potential information corresponding to the plurality of pieces of label information. The deduction part 104 acquires the potential information related to the designated label information from the plurality of pieces of potential information (S404) and outputs the potential information (S106).

In this case, the inferring device 1 may receive input of the label information as above and perform output based on the label information As another example, the inferring device 1 may accept or does not need to accept the input related to the label information, and may output the pieces of potential information related to the plurality of pieces of label information via the output part 106.

The model NN is configured to generate an output with respect to the first condition and an output with respect to the second condition. Further, the model NN is trained based on the first label information to output the first output with respect to the first condition and the second output with respect to the second condition, and is used in the inferring device 1.

The use of the model NN trained as above makes it possible for the inferring device 1 to acquire a deduction result of the potential information obtained by an arithmetic operation based on the label information corresponding to the node from the node of the output layer when the atomic structure is input. The label information may be set, for example, by defining the mode similar to that defined in the above embodiment. This configuration makes expansion easier than in the other configuration when executing re-training while increasing the label information with respect to an already existing trained model.

As explained above, it becomes possible to appropriately change the input/output of the model NN, specifically, the node or layer which receives input of the label information.

Note that though a first . . . and a second . . . are used for explanation also in this embodiment as in the above embodiment, there may be a third . . . , a fourth . . . , and so on as a matter of course. The training and deduction can be executed based on the plurality of conditions. This also applies to a third embodiment illustrated below.

### Third Embodiment

The atomic structure may be converted into a common intermediate representation based on the label information, and the intermediate representation may be input into the model NN.

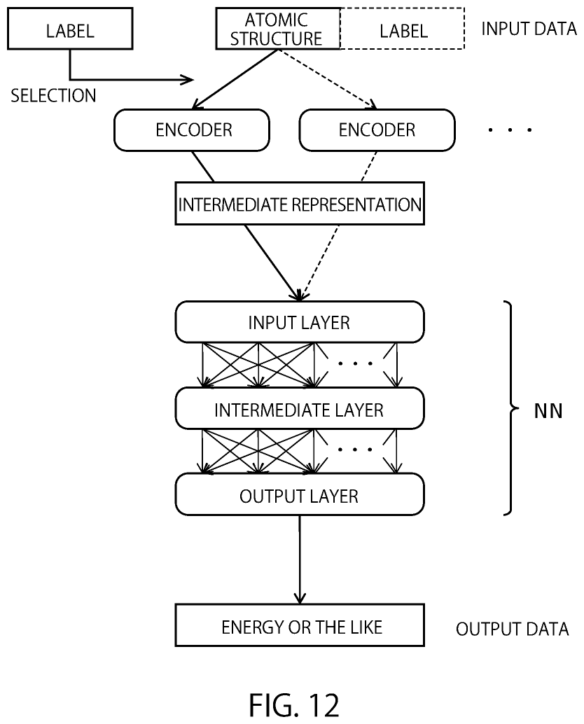

FIG. 12 is a chart schematically illustrating a model according to this embodiment. As illustrated in FIG. 12, the atomic structure is first input into an encoder, and an output from the encoder is converted into the intermediate representation. The intermediate representation may be input into the model NN. The encoder can be an arbitrary neural network model as long as it can realize appropriate conversion.

The training device 2 may define the encoder at a granularity for each piece of label information, for example, for each piece of software or for each mode. The training part 204 designates the encoder into which the atomic structure is input based on the label information, and inputs the atomic structure into the designated encoder. Then, the training part 204 inputs the output from the encoder into the model NN, and executes the training of the model NN as in each of the above embodiments. In this embodiment, the training of the encoder is performed together with the training of the model NN. In other words, the training part 204 updates the parameter up to the input layer by the error backward propagation based on the output from the model NN, and continuously executes update of the parameter of the encoder using the gradient information backward propagated to the input layer. The training is repeated in this manner.

As explained above, the same or different encoder and one model NN are trained for each piece of label information.

In the inferring device 1, a plurality of trained encoders and one model NN which have been trained as above are used. When the atomic structure and the label information are input, the deduction part 104 of the inferring device 1 first selects the encoder which converts into an intermediate representation based on the label information, and converts the atomic structure into an intermediate representation.

Subsequently, the deduction part 104 inputs the intermediate representation into the model NN and forward propagates it to infer the potential information. This deduction has already acquired an intermediate representation in consideration of the label information in the encoder at the preceding stage, and makes it possible to acquire the potential information from the atomic structure as an arithmetic result appropriately based on the label information.

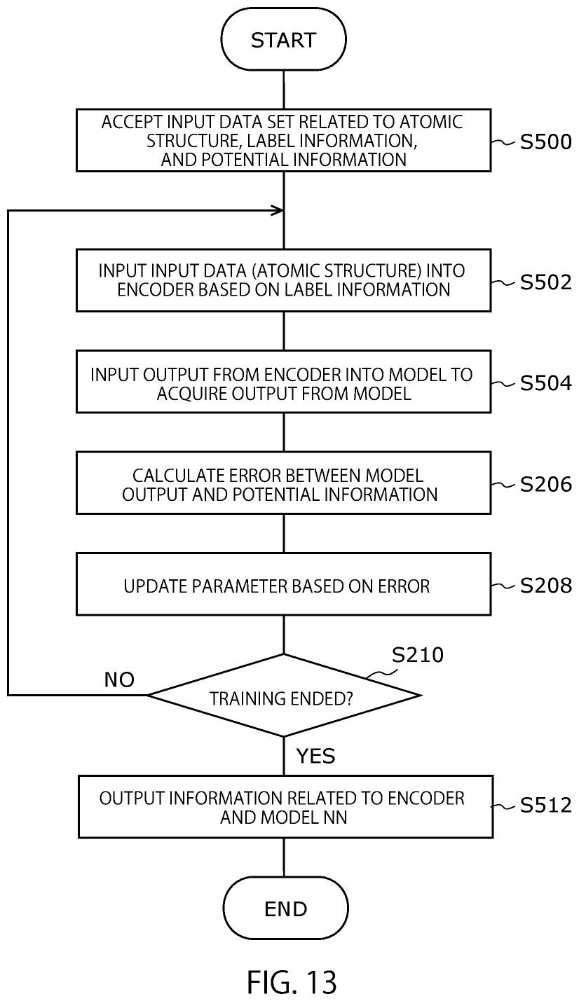

FIG. 13 is a flowchart illustrating processing of training by the training device 2 in the configuration of FIG. 12. The same code as that in FIG. 6 represents the same processing unless otherwise stated.

The training part 204 inputs, after acquiring the input data, the data related to the atomic structure into the encoder based on the label information to acquire an output from the encoder (S502). The output from the encoder may be, for example, a variable obtained by dimensional compression (dimensional reduction) of the atomic structure based on the label information.

The training part 204 inputs the output from the encoder selected by the label information into the model NN to acquire an output from the model NN (S504). After the processing at S206, the training part 204 backward propagates the error between the output from the model NN and the potential information to update the parameters of the model NN and the encoder selected based on the label information (S208).

Until the training satisfies the end condition (S210: NO), the processing at S502 to S208 is repeated, and when the training is ended (S210: YES), the training device 2 outputs information related to the encoder and the model NN2 (S512) and ends the processing.

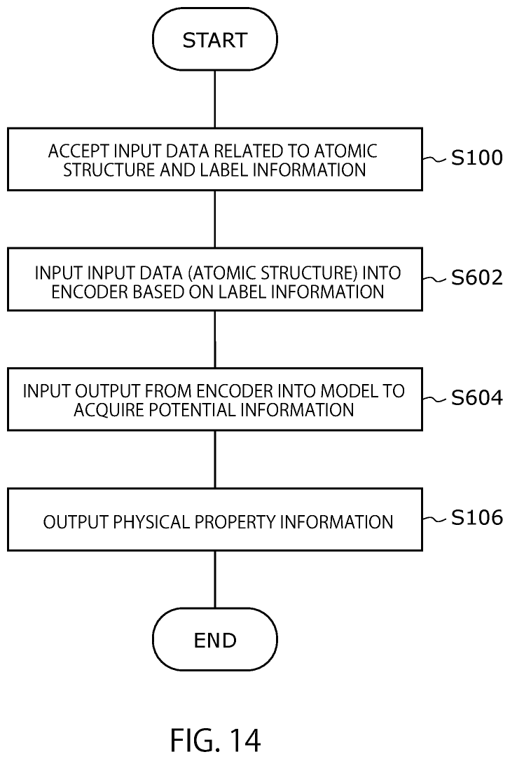

FIG. 14 is a flowchart illustrating processing of training by the inferring device 1 in the configuration in FIG. 12. The same code as that in FIG. 3 represents the same processing.

The deduction part 104 selects an encoder based on the label information, and inputs the input data into the encoder to acquire an output from the encoder (S602).

Subsequently, the deduction part 104 inputs the output from the encoder into the model NN to acquire potential information (S604). The inferring device 1 outputs the potential information.

As explained above, the plurality of encoders and the model NN are trained so as to receive input of the information related to the first atomic structure into the encoder (first neural network model) decided based on the first label information and input its output into the model NN to acquire a first output, and receive input of the information related to the second atomic structure into the encoder (second neural network model) decided based on the second label information and input its output into the model NN to acquire a second output, and are used in the inferring device 1.

Note that as illustrated in the drawings, all of the label information does not need to be used for the selection of the encoder. In other words, training may be executed by selecting the encoder using information being a part of the label information (for example, software) and inputting information on the remaining label information (for example, arithmetic parameter) into the designated encoder together with the atomic structure. In this case, the label information to be input into the encoder may vary depending on the selected encoder. As a result of this, it is possible to delete extra nodes in the input of the encoder, and it is also possible to more appropriately realize the conversion from the encoder to the intermediate representation, namely, the addition of the label information to the atomic structure.

As explained above, according to this embodiment, the use of the common intermediate representation for input into the model NN makes it possible to train the model for acquiring the potential information in which the label information is appropriately reflected, and realize deduction using the model.

As in the case of FIG. 9, the configuration of such a neural network model can improve the scalability in the case of performing re-training such as increasing the label information with respect to the trained model NN.

The trained models of above embodiments may be, for example, a concept that includes a model that has been trained as described and then distilled by a general method.

Some or all of each device (the inference device 1 or the training device 2) in the above embodiment may be configured in hardware, or information processing of software (program) executed by, for example, a CPU (Central Processing Unit), GPU (Graphics Processing Unit). In the case of the information processing of software, software that enables at least some of the functions of each device in the above embodiments may be stored in a non-volatile storage medium (non-volatile computer readable medium) such as CD-ROM (Compact Disc Read Only Memory) or USB (Universal Serial Bus) memory, and the information processing of software may be executed by loading the software into a computer. In addition, the software may also be downloaded through a communication network. Further, entire or a part of the software may be implemented in a circuit such as an ASIC (Application Specific Integrated Circuit) or FPGA (Field Programmable Gate Array), wherein the information processing of the software may be executed by hardware.

A storage medium to store the software may be a removable storage media such as an optical disk, or a fixed type storage medium such as a hard disk, or a memory. The storage medium may be provided inside the computer (a main storage device or an auxiliary storage device) or outside the computer.

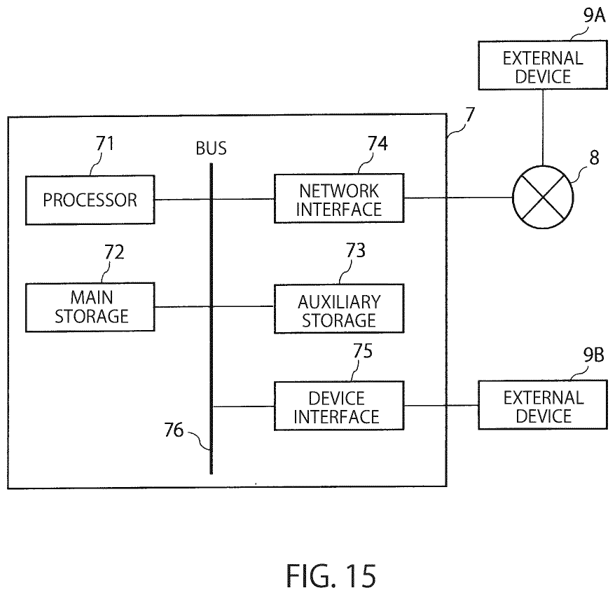

FIG. 15 is a block diagram illustrating an example of a hardware configuration of each device (the inference device 1 or the training device 2) in the above embodiments. As an example, each device may be implemented as a computer 7 provided with a processor 71, a main storage device 72, an auxiliary storage device 73, a network interface 74, and a device interface 75, which are connected via a bus 76.

The computer 7 of FIG. 15 is provided with each component one by one but may be provided with a plurality of the same components. Although one computer 7 is illustrated in FIG. 15, the software may be installed on a plurality of computers, and each of the plurality of computer may execute the same or a different part of the software processing. In this case, it may be in a form of distributed computing where each of the computers communicates with each of the computers through, for example, the network interface 74 to execute the processing. That is, each device (the inference device 1 or the training device 2) in the above embodiments may be configured as a system where one or more computers execute the instructions stored in one or more storages to enable functions. Each device may be configured such that the information transmitted from a terminal is processed by one or more computers provided on a cloud and results of the processing are transmitted to the terminal.

Various arithmetic operations of each device (the inference device 1 or the training device 2) in the above embodiments may be executed in parallel processing using one or more processors or using a plurality of computers over a network. The various arithmetic operations may be allocated to a plurality of arithmetic cores in the processor and executed in parallel processing. Some or all the processes, means, or the like of the present disclosure may be implemented by at least one of the processors or the storage devices provided on a cloud that can communicate with the computer 7 via a network. Thus, each device in the above embodiments may be in a form of parallel computing by one or more computers.

The processor 71 may be an electronic circuit (such as, for example, a processor, processing circuitry, processing circuitry, CPU, GPU, FPGA, or ASIC) that executes at least controlling the computer or arithmetic calculations. The processor 71 may also be, for example, a general-purpose processing circuit, a dedicated processing circuit designed to perform specific operations, or a semiconductor device which includes both the general-purpose processing circuit and the dedicated processing circuit. Further, the processor 71 may also include, for example, an optical circuit or an arithmetic function based on quantum computing.

The processor 71 may execute an arithmetic processing based on data and/or a software input from, for example, each device of the internal configuration of the computer 7, and may output an arithmetic result and a control signal, for example, to each device. The processor 71 may control each component of the computer 7 by executing, for example, an OS (Operating System), or an application of the computer 7.

Each device (the inference device 1 or the training device 2) in the above embodiments may be enabled by one or more processors 71. The processor 71 may refer to one or more electronic circuits located on one chip, or one or more electronic circuitries arranged on two or more chips or devices. In the case of a plurality of electronic circuitries are used, each electronic circuit may communicate by wired or wireless.

The main storage device 72 may store, for example, instructions to be executed by the processor 71 or various data, and the information stored in the main storage device 72 may be read out by the processor 71. The auxiliary storage device 73 is a storage device other than the main storage device 72. These storage devices shall mean any electronic component capable of storing electronic information and may be a semiconductor memory. The semiconductor memory may be either a volatile or non-volatile memory. The storage device for storing various data or the like in each device (the inference device 1 or the training device 2) in the above embodiments may be enabled by the main storage device 72 or the auxiliary storage device 73 or may be implemented by a built-in memory built into the processor 71. For example, the storages 102, 202 in the above embodiments may be implemented in the main storage device 72 or the auxiliary storage device 73.

In the case of each device (the inference device 1 or the training device 2) in the above embodiments is configured by at least one storage device (memory) and at least one of a plurality of processors connected/coupled to/with this at least one storage device, at least one of the plurality of processors may be connected to a single storage device. Or at least one of the plurality of storages may be connected to a single processor. Or each device may include a configuration where at least one of the plurality of processors is connected to at least one of the plurality of storage devices. Further, this configuration may be implemented by a storage device and a processor included in a plurality of computers. Moreover, each device may include a configuration where a storage device is integrated with a processor (for example, a cache memory including an L1 cache or an L2 cache).

The network interface 74 is an interface for connecting to a communication network 8 by wireless or wired. The network interface 74 may be an appropriate interface such as an interface compatible with existing communication standards. With the network interface 74, information may be exchanged with an external device 9A connected via the communication network 8. Note that the communication network 8 may be, for example, configured as WAN (Wide Area Network), LAN (Local Area Network), or PAN (Personal Area Network), or a combination of thereof, and may be such that information can be exchanged between the computer 7 and the external device 9A. The internet is an example of WAN, IEEE802.11 or Ethernet (registered trademark) is an example of LAN, and Bluetooth (registered trademark) or NFC (Near Field Communication) is an example of PAN.

The device interface 75 is an interface such as, for example, a USB that directly connects to the external device 9B.

The external device 9A is a device connected to the computer 7 via a network. The external device 9B is a device directly connected to the computer 7.

The external device 9A or the external device 9B may be, as an example, an input device. The input device is, for example, a device such as a camera, a microphone, a motion capture, at least one of various sensors, a keyboard, a mouse, or a touch panel, and gives the acquired information to the computer 7. Further, it may be a device including an input unit such as a personal computer, a tablet terminal, or a smartphone, which may have an input unit, a memory, and a processor.

The external device 9A or the external device 9B may be, as an example, an output device. The output device may be, for example, a display device such as, for example, an LCD (Liquid Crystal Display), or an organic EL (Electro Luminescence) panel, or a speaker which outputs audio. Moreover, it may be a device including an output unit such as, for example, a personal computer, a tablet terminal, or a smartphone, which may have an output unit, a memory, and a processor.

Further, the external device 9A or the external device 9B may be a storage device (memory). The external device 9A may be, for example, a network storage device, and the external device 9B may be, for example, an HDD storage.

Furthermore, the external device 9A or the external device 9B may be a device that has at least one function of the configuration element of each device (the inference device 1 or the training device 2) in the above embodiments. That is, the computer 7 may transmit a part of or all of processing results to the external device 9A or the external device 9B, or receive a part of or all of processing results from the external device 9A or the external device 9B.

In the present specification (including the claims), the representation (including similar expressions) of “at least one of a, b, and c” or “at least one of a, b, or c” includes any combinations of a, b, c, a-b, a-c, b-c, and a-b-c. It also covers combinations with multiple instances of any element such as, for example, a-a, a-b-b, or a-a-b-b-c-c. It further covers, for example, adding another element d beyond a, b, and/or c, such that a-b-c-d.

In the present specification (including the claims), the expressions such as, for example, “data as input,” “using data,” “based on data,” “according to data,” or “in accordance with data” (including similar expressions) are used, unless otherwise specified, this includes cases where data itself is used, or the cases where data is processed in some ways (for example, noise added data, normalized data, feature quantities extracted from the data, or intermediate representation of the data) are used. When it is stated that some results can be obtained “by inputting data,” “by using data,” “based on data,” “according to data,” “in accordance with data” (including similar expressions), unless otherwise specified, this may include cases where the result is obtained based only on the data, and may also include cases where the result is obtained by being affected factors, conditions, and/or states, or the like by other data than the data. When it is stated that “output/outputting data” (including similar expressions), unless otherwise specified, this also includes cases where the data itself is used as output, or the cases where the data is processed in some ways (for example, the data added noise, the data normalized, feature quantity extracted from the data, or intermediate representation of the data) is used as the output.

In the present specification (including the claims), when the terms such as “connected (connection)” and “coupled (coupling)” are used, they are intended as non-limiting terms that include any of “direct connection/coupling,” “indirect connection/coupling,” “electrically connection/coupling,” “communicatively connection/coupling,” “operatively connection/coupling,” “physically connection/coupling,” or the like. The terms should be interpreted accordingly, depending on the context in which they are used, but any forms of connection/coupling that are not intentionally or naturally excluded should be construed as included in the terms and interpreted in a non-exclusive manner.

In the present specification (including the claims), when the expression such as “A configured to B,” this may include that a physically structure of A has a configuration that can execute operation B, as well as a permanent or a temporary setting/configuration of element A is configured/set to actually execute operation B. For example, when the element A is a general-purpose processor, the processor may have a hardware configuration capable of executing the operation B and may be configured to actually execute the operation B by setting the permanent or the temporary program (instructions). Moreover, when the element A is a dedicated processor, a dedicated arithmetic circuit, or the like, a circuit structure of the processor or the like may be implemented to actually execute the operation B, irrespective of whether or not control instructions and data are actually attached thereto.

In the present specification (including the claims), when a term referring to inclusion or possession (for example, “comprising/including,” “having,” or the like) is used, it is intended as an open-ended term, including the case of inclusion or possession an object other than the object indicated by the object of the term. If the object of these terms implying inclusion or possession is an expression that does not specify a quantity or suggests a singular number (an expression with a or an article), the expression should be construed as not being limited to a specific number.

In the present specification (including the claims), although when the expression such as “one or more,” “at least one,” or the like is used in some places, and the expression that does not specify a quantity or suggests a singular number (the expression with a or an article) is used elsewhere, it is not intended that this expression means “one.” In general, the expression that does not specify a quantity or suggests a singular number (the expression with a or an as article) should be interpreted as not necessarily limited to a specific number.

In the present specification, when it is stated that a particular configuration of an example results in a particular effect (advantage/result), unless there are some other reasons, it should be understood that the effect is also obtained for one or more other embodiments having the configuration. However, it should be understood that the presence or absence of such an effect generally depends on various factors, conditions, and/or states, etc., and that such an effect is not always achieved by the configuration. The effect is merely achieved by the configuration in the embodiments when various factors, conditions, and/or states, etc., are met, but the effect is not always obtained in the claimed invention that defines the configuration or a similar configuration.

In the present specification (including the claims), when the term such as “maximize/maximization” is used, this includes finding a global maximum value, finding an approximate value of the global maximum value, finding a local maximum value, and finding an approximate value of the local maximum value, should be interpreted as appropriate accordingly depending on the context in which the term is used. It also includes finding on the approximated value of these maximum values probabilistically or heuristically. Similarly, when the term such as “minimize/minimization” is used, this includes finding a global minimum value, finding an approximated value of the global minimum value, finding a local minimum value, and finding an approximated value of the local minimum value, and should be interpreted as appropriate accordingly depending on the context in which the term is used. It also includes finding the approximated value of these minimum values probabilistically or heuristically. Similarly, when the term such as “optimize/optimization” is used, this includes finding a global optimum value, finding an approximated value of the global optimum value, finding a local optimum value, and finding an approximated value of the local optimum value, and should be interpreted as appropriate accordingly depending on the context in which the term is used. It also includes finding the approximated value of these optimal values probabilistically or heuristically.

In the present specification (including claims), when a plurality of hardware performs a predetermined process, the respective hardware may cooperate to perform the predetermined process, or some hardware may perform all the predetermined process. Further, a part of the hardware may perform a part of the predetermined process, and the other hardware may perform the rest of the predetermined process. In the present specification (including claims), when an expression (including similar expressions) such as “one or more hardware perform a first process and the one or more hardware perform a second process,” or the like, is used, the hardware that perform the first process and the hardware that perform the second process may be the same hardware, or may be the different hardware. That is: the hardware that perform the first process and the hardware that perform the second process may be included in the one or more hardware. Note that, the hardware may include an electronic circuit, a device including the electronic circuit, or the like.

While certain embodiments of the present disclosure have been described in detail above, the present disclosure is not limited to the individual embodiments described above. Various additions, changes, substitutions, partial deletions, etc. are possible to the extent that they do not deviate from the conceptual idea and purpose of the present disclosure derived from the contents specified in the claims and their equivalents. For example, when numerical values or mathematical formulas are used in the description in the above-described embodiments, they are shown for illustrative purposes only and do not limit the scope of the present disclosure. Further, the order of each operation shown in the embodiments is also an example, and does not limit the scope of the present disclosure.

1. An inferring device comprising:
at least one memory; and
at least one processor configured to:
acquire an output from a neural network model based on information related to an atomic structure and label information related to an atomic simulation, wherein
the neural network model is trained to infer a simulation result with respect to the atomic structure generated by the atomic simulation corresponding to the label information.

2. The inferring device according to claim 1, wherein
the at least one processor is configured to:
acquire the output by inputting the information related to the atomic structure and the label information into the neural network model.

3. The inferring device according to claim 2, wherein
the at least one processor is configured to:
input the label information into at least one of an intermediate layer or an output layer of the neural network model.

4. The inferring device according to claim 1, wherein:
the neural network model is configured to generate a plurality of outputs; and
the at least one processor is configured to:
acquire the output by selecting one of the plurality of outputs from the neural network model based on the label information.

5. The inferring device according to claim 1, wherein
the at least one processor is configured to:
input the information related to the atomic structure into a first neural network model decided based on the label information; and
acquire the output by inputting an output from the first neural network model into the neural network model.

6. The inferring device according to claim 1, wherein
the label information includes at least one of
information on software to be used for the atomic simulation,
information on a calculation technique to be used for the atomic simulation,
information on a function to be used for the atomic simulation,
information on a parameter to be used for the atomic simulation,
information on a calculation condition to be used for the atomic simulation, or
information on an arithmetic mode to be used for the atomic simulation.

7. The inferring device according to claim 1, wherein
the neural network model is a model relating to NNP (Neural Network Potential).

8. The inferring device according to claim 1, wherein
the atomic simulation is executed using a first-principles calculation.

9. The inferring device according to claim 1, wherein
the atomic simulation is executed using a DFT (Density Function Theory) calculation.

10. The inferring device according to claim 9, wherein
the label information includes at least one of information on a functional or information on a basis function.

11. The inferring device according to claim 9, wherein:
the label information includes at least one of information on a first condition of the DFT calculation or information on a second condition of the DFT calculation;
the first condition is a condition under which the DFT calculation higher in accuracy than under the second condition is executable, under a periodic boundary condition; and
the second condition is a condition under which the DFT calculation higher in accuracy than under the first condition is executable, under a free boundary condition.

12. The inferring device according to claim 1, wherein
the atomic simulation is at least one of
a simulation to be executed using two or more different pieces of software,
a simulation to be executed using two or more different calculation techniques,
a simulation to be executed using two or more different functions,
a simulation to be executed using two or more different parameters,
a simulation to be executed using two or more different calculation conditions, or
a simulation to be executed using two or more different arithmetic modes.

13. A training device comprising:
at least one memory; and
at least one processor configured to:
acquire a first output from a neural network model based on information related to a first atomic structure and first label information related to an atomic simulation;
calculate first difference information being a difference between the first output and a first simulation result with respect to the first atomic structure generated by the atomic simulation corresponding to the first label information;
acquire a second output from the neural network model based on information related to a second atomic structure and second label information related to the atomic simulation;
calculate second difference information being a difference between the second output and a second simulation result with respect to the second atomic structure generated by the atomic simulation corresponding to the second label information; and
update a parameter of the neural network model based on the first difference information and the second difference information.

14. The training device according to claim 13, wherein:
the at least one processor is configured to:
acquire the first output by inputting the information related to the first atomic structure and the first label information into the neural network model; and
acquire the second output by inputting the information related to the second atomic structure and the second label information into the neural network model.

15. The training device according to claim 13, wherein:
the neural network model is configured to generate an output with respect to the first label information and an output with respect to the second label information; and
the at least one processor is configured to:
acquire the output with respect to the first label information as the first output based on the first label information; and
acquire the output with respect to the second label information as the second output based on the second label information.

16. The training device according to claim 13, wherein
the at least one processor is configured to:
input the information related to the first atomic structure into a first neural network model decided based on the first label information;
acquire the first output by inputting an output from the first neural network model into the neural network model;
input the information related to the second atomic structure into a second neural network model decided based on the second label information;
acquire the second output by inputting an output from the second neural network model into the neural network model;
update a parameter of the first neural network model based on the first difference information, and
update a parameter of the second neural network model based on the second difference information.

17. The training device according to claim 13, wherein
the first atomic structure and the second atomic structure include the same or almost the same atomic structure.

18. The training device according to claim 13, wherein:
the atomic simulation is executed using two or more different pieces of software; and
the atomic simulation corresponding to the first label information and the atomic simulation corresponding to the second label information are executed using the same software or different pieces of software.

19. The training device according to claim 13, wherein
the atomic simulation is at least one of
a simulation to be executed using two or more different pieces of software,
a simulation to be executed using two or more different calculation techniques,
a simulation to be executed using two or more different functions;
a simulation to be executed using two or more different parameters,
a simulation to be executed using two or more different calculation conditions, or
a simulation to be executed using two or more different arithmetic modes.

20. The training device according to claim 13, wherein:
the first label information includes at least one of
information on first software to be used for the atomic simulation,
information on a first calculation technique to be used for the atomic simulation,
information on a first function to be used for the atomic simulation,
information on a first parameter to be used for the atomic simulation,
information on a first calculation condition to be used for the atomic simulation, or
information on a first arithmetic mode to be used for the atomic simulation; and
the second label information includes at least one of
information on second software to be used for the atomic simulation,
information on a second calculation technique to be used for the atomic simulation,
information on a second function to be used for the atomic simulation,
information on a second parameter to be used for the atomic simulation,
information on a second calculation condition to be used for the atomic simulation, or
information on a second arithmetic mode to be used for the atomic simulation.

21. The training device according to claim 13, wherein
the neural network model is a model relating to NNP.

22. The training device according to claim 13, wherein
the atomic simulation is executed using a first-principles calculation.

23. The training device according to claim 13, wherein
the atomic simulation is executed using a DFT calculation.

24. The training device according to claim 23, wherein:
the first label information includes at least one of information on a first functional or information on a first basis function; and
the second label information includes at least one of information on a second functional or information on a second basis function.

25. The training device according to claim 23, wherein:
the first label information includes information on a first condition of the DFT calculation;
the second label information includes information on a second condition of the DFT calculation;
the first condition is a condition under which the DFT calculation higher in accuracy than under the second condition is executable, under a periodic boundary condition; and
the second condition is a condition under which the DFT calculation higher in accuracy than under the first condition is executable, under a free boundary condition.

26. A method for inferring by one or more processors, comprising:
acquiring an output from a neural network model based on information related to an atomic structure and label information related to an atomic simulation, wherein
the neural network model is trained to infer a simulation result with respect to the atomic structure generated by the atomic simulation corresponding to the label information.

27. The method according to claim 26 further comprising inputting the information related to the atomic structure and the label information into the neural network model to acquire the output.

28. The method according to claim 26, wherein
the label information includes at least one of
information on software to be used for the atomic simulation,
information on a calculation technique to be used for the atomic simulation,
information on a function to be used for the atomic simulation,
information on a parameter to be used for the atomic simulation,
information on a calculation condition to be used for the atomic simulation, or
information on an arithmetic mode to be used for the atomic simulation.

29. The method according to claim 26, wherein
the atomic simulation is executed using a DFT.

30. The method according to claim 29, wherein
the label information includes at least one of information on a functional or information on a basis function.

31. The method according to claim 29, wherein
the label information includes at least one of information on a first condition of the DFT calculation or information on a second condition of the DFT calculation;
the first condition is a condition under which the DFT calculation higher in accuracy than under the second condition is executable, under a periodic boundary condition; and
the second condition is a condition under which the DFT calculation higher in accuracy than under the first condition is executable, under a free boundary condition.

32. The method according to claim 26, wherein
the atomic simulation is at least one of
a simulation to be executed using two or more different pieces of software,
a simulation to be executed using two or more different calculation techniques,
a simulation to be executed using two or more different functions,
a simulation to be executed using two or more different parameters,
a simulation to be executed using two or more different calculation conditions, or
a simulation to be executed using two or more different arithmetic modes.
